(19) United States
(12) Patent Application Publication
(10) Pub. No.: US 2025/0388857 A1
(43) Pub. Date: Dec. 25, 2025

(54) USE OF TRIBUTYRIN AS AN ADDITIVE FOR EMBRYONIC DEVELOPMENT CULTURE MEDIUM IN VITRO

(71) Applicant: Institute of Animal Sciences and Veterinary Medicine, Hubei Academy of Agricultural Sciences, Wuhan (CN)

(72) Inventors: Fan Chen, Wuhan (CN); Lixiang Zheng, Wuhan (CN); Anfeng Luo, Wuhan (CN); Yanzhen Bi, Wuhan (CN); Hongyan Ren, Wuhan (CN); Hao Gu, Wuhan (CN); Changfan Zhou, Wuhan (CN); Wei Zeng, Wuhan (CN)

(21) Appl. No.: 19/186,774

(22) Filed: Apr. 23, 2025

(30) Foreign Application Priority Data
Jun. 24, 2024 (CN) 202410820667X

(51) Int. Cl. C12N 5/073; C12N 5/00

(52) U.S. Cl. CPC C12N 5/0604; C12N 5/0018; C12N 2501/999

(57) ABSTRACT

The present discloses provides the use of tributyrin as an additive for embryonic development culture medium in vitro, in the present disclosure, the tributyrin is applied as an additive to the development culture medium of mouse embryos in vitro for the first time, the tributyrin can significantly increase the rate of blastocyst, and reduce the ROS content in the embryos, improve the mitochondrial membrane potential in the embryos, increase the ATP level and the expression of antioxidant genes in the embryos, improve the DNA methylation and histone modification level in the embryos, promote the embryos development in vitro; In addition, tributyrin, as natural antioxidant and apparent drug, which is safe, non-toxic and side effects; the tributyrin provides strong support for the efficient embryos development of human assisted reproductive technology, mammalian fertilization embryos, parthenogenetic embryos and somatic cell cloned embryos and other embryo engineering technologies in vitro.

### FIELD OF THE DISCLOSURE

The present disclosure belongs to the field of animal reproductive biology technology, and specifically relates to the use of tributyrin as an additive for embryonic development culture medium in vitro.

### BACKGROUND

One of the important issues faced during the embryo in vitro culture (IVC) is the environment, culture medium, mineral oil, and the production and accumulation of reactive oxygen species (ROS) of the embryo itself. Inadequate ROS clearance induce cellular oxidative stress, which severely reduces the developmental capacity of embryos through the oxidation of RNA, DNA, and proteins. Unlike in vitro culture, somatic cells in the in vivo environment are able to consume oxygen, providing a hypoxic environment for gamete and early embryonic development; Moreover, the body's highly effective antioxidant system can remove freshly produced ROS in time to protect cells from ROS damage. Compared to the in vivo environment, the antioxidative capacity of embryos in vitro was reduced, making them more susceptible to oxidative stress and leading to elevated ROS levels. Therefore, in order to improve the developmental capacity of in vitro embryos, a variety of antioxidants are applied to IVC.

In the early stages of embryonic development, the transcriptional level of the zygotic genome is silent, and development is dependent on the maternal materials stored in the oocyte. With the progress of embryonic development, the maternal materials will be gradually degraded, and the zygotic genome will gradually activate transcription, marking the shift of embryonic development from maternal regulation to zygotic genome regulation. During zygotic genome activation (ZGA), epigenetic modifications on the genome are reprogrammed on a large scale, and the developmental arrest of embryos in vitro first occurs at the ZGA stage, largely due to the failure of some key epigenetics to be completely reprogrammed by oocytes, among which DNA and histone epigenetic modifications are the most important. Embryos in vitro of a variety of animals have been found to have abnormal levels of DNA methylation and histone acetylation at the ZGA stage, resulting in insufficient ZGA initiation and delayed embryonic development. Thus, epigenetic disorders are an important barrier that hinders the development of embryos in vitro. Therefore, in order to improve the developmental capacity of embryos in vitro, a variety of epigenetic modification drugs have been tried to be applied to IVC.

Tributyrin (TB) is an ester compound formed from glycerol and butyric acid, which has a variety of biological activities and pharmacological properties, so it has attracted attention and research. However, at present, there are no studies on the use of tributyrin in embryonic development in vitro.

### SUMMARY

The object of the present disclosure is to provide the use of tributyrin as an additive for embryonic development culture medium in vitro. In the present disclosure, the tributyrin is applied as an additive to the development culture medium of mouse embryos in vitro for the first time, the tributyrin can significantly increase the rate of blastocyst, and reduce the ROS content in the embryos, improve the mitochondrial membrane potential in the embryos, increase the ATP level and the expression of antioxidant genes in the embryos, improve the DNA methylation and histone modification level in the embryos, promote the embryos development in vitro.

In the first aspect, the present disclosure provides the use of tributyrin as an additive for embryonic development culture medium in vitro, the use including promoting embryos development.

In some embodiments, promoting embryos development including any of the following: A1) reduce the ROS content in the embryos; A2) improve the mitochondrial membrane potential in the embryos; A3) increase the ATP level and the expression of antioxidant genes in the embryos; A4) improve the DNA methylation and histone modification level in the embryos.

In some embodiments, the concentration of tributyrin is 50˜200 μmol/L, for example, it can be 50 μmol/L, 80 μmol/L, 100 μmol/L, 120 μmol/L, 150 μmol/L, 160 μmol/L, 180 μmol/L, 200 μmol/L, or other values in the range.

In some embodiments, the concentration of tributyrin is 100 μmol/L.

In some embodiments, embryonic development culture medium in vitro including KMSO basal culture medium.

In the second aspect, the present disclosure provides an additive for embryonic development culture medium in vitro to promote embryos development, the additive comprising tributyrin.

In the third aspect, the present disclosure provides an embryonic development culture medium in vitro for promoting embryos development, the culture medium comprising the above-mentioned additive.

In some embodiments, the concentration of additive is 50˜200 μmol/L, for example, it can be 50 μmol/L, 80 μmol/L, 100 μmol/L, 120 μmol/L, 150 μmol/L, 160 μmol/L, 180 μmol/L, 200 μmol/L, or other values in the range.

In some embodiments, the concentration of the additive is 100 μmol/L.

In some embodiments, the culture medium also including KMSO basal culture medium.

The beneficial effect of the present disclosure is: different from the case of prior art, in the present disclosure, the tributyrin is applied as an additive to the development culture medium of mouse embryos in vitro for the first time, the tributyrin can significantly increase the rate of blastocyst, and reduce the ROS content in the embryos, improve the mitochondrial membrane potential in the embryos, increase the ATP level and the expression of antioxidant genes in the embryos, improve the DNA methylation and histone modification level in the embryos, promote the embryos development in vitro; In addition, tributyrin, as natural antioxidant and apparent drug, which is safe, non-toxic and side effects, cheap and easy to obtain, and does not need to replace the culture medium in the whole culture process, and is simple to operate; the tributyrin provides strong support for the efficient embryos development of human assisted reproductive technology, mammalian fertilization embryos, parthenogenetic embryos and somatic cell cloned embryos and other embryo engineering technologies in vitro, and has good application prospects.

### BRIEF DESCRIPTION OF THE DRAWINGS

FIG. 1 is the result of tributyrin on blastocyst formation rate of mouse in different treatment groups of example 2 of the present disclosure, wherein, figure (A) is the microscopic observation result of mouse embryos, figure (B) is the result of the formation rate of mouse blastocyst;

FIG. 2 is the result of the effect of tributyrin on ROS level in mouse embryos in different treatment groups of example 3 of the present disclosure, wherein, figure (A) is the result of ROS fluorescence in mouse embryos, and figure (B) is the result of ROS level in mouse embryos;

FIG. 3 is the result of the effect of tributyrin on mitochondrial membrane potential in mouse embryos in different treatment groups of example 3 of the present disclosure, wherein, figure (A) is the result of J C-1 staining fluorescence in mouse embryos, and figure (B) is the result of J C-1 red/J C-1 green level in mouse embryos;

FIG. 4 is the result of the effect of tributyrin on ATP level in mouse embryos in different treatment groups of example 3 of the present disclosure;

FIG. 5 is the result of the effect of tributyrin on expression level of antioxidant-related genes in mouse embryos in different treatment groups of example 3 of the present disclosure;

FIG. 6 is the result of the effect of tributyrin on 5 hmC level in mouse embryonic genome in different treatment groups of example 3 of the present disclosure, wherein, figure (A) is the result of 5 hmC fluorescence in mouse embryos, and figure (B) is the result of 5 hmC fluorescence intensity in mouse embryos;

FIG. 7 is the result of the effect of tributyrin on the level of histone H3K27ac modification in mouse embryonic genome in different treatment groups of example 3 of the present disclosure, wherein, figure (A) is the result of H3K27ac fluorescence in mouse embryos, and figure (B) is the result of H3K27ac fluorescence intensity in mouse embryos.

### DETAILED DESCRIPTION OF PREFERRED EMBODIMENTS

The technical solutions in the examples of the present disclosure will be clearly and completely described below in conjunction with the examples of the present disclosure, and it is obvious that the described examples are only part examples of the present disclosure, not all examples. Based on the examples in the present disclosure, all other examples obtained by a person skilled in the art without making creative work belong to the scope of protection of the present disclosure.

The experimental methods that do not specify the specific conditions in the examples are usually in accordance with the general conditions and the conditions described in the manual, or in accordance with the conditions recommended by the manufacturer, and the general equipment, materials, reagents, etc., used can generally be obtained commercially unless otherwise specified.

### Example 1 Preparation of Development Culture Medium In Vitro of Mouse Embryos

The preparation method of the development culture medium in vitro of the mouse embryos is as follows:

(1) Preparation of tributyrin stock solution: 87.8 μL of tributyrin (purchased from Sigma, catalog number: 91010) was dissolved in 1 mL of DMSO, tributyrin stock solution with the concentration of 300 mmol/L was obtained, which was divided into 5 μL per tube, and stored at −20° C.;

(2) The tributyrin stock solution prepared in step (1) was added to the KSOM basic embryo culture medium (purchased from Sigma, catalog number: MR-101-D) to obtain the development culture medium in vitro of mouse embryos.
wherein, the culture medium was divided into the following two groups for subsequent experimental tests:

Basal development culture medium (control group): KSOM basal embryo culture medium;

Development culture medium of adding tributyrin (treatment group): based on KSOM basal culture medium, 50 μmol/L or 100 μmol/L or 200 μmol/L of tributyrin was supplemented (use immediately after preparation).

### Example 2 Effect of Tributyrin on the Development of Mouse Embryos In Vitro

Specifically, at 4:00 p.m. on the first day, 10 IU pregnant horse serum gonadotropin was injected intraperitoneally into Kunming female mouse with SPF level for 6˜9 weeks, and 8 IU chorionic gonadotropin was injected after 48 hours. At 9:00 the next day, the 9˜12-week male mouse was killed by cervical dislocation, and the tail of the epididymis next to the vas deferens was taken, after cutting with ophthalmic scissors in 200 μL of insemination droplet (Sigma, MR-070-D) is to release sperm for 60 minutes. The insemination droplet need to be pre-balanced the night before. At the same time, 4 female mouse were killed by neck detachment, the enlargement of oviduct was disposed of in the sperm solution, the insulin needle was used to open the ampulla side under the microscope, and the cumulus-oocyte complex (COCs) would be released from the ampulla under the action of squeezing or internal pressure. 8˜10 μL of sperm and COCs were taken in the insemination droplet. After 6 h after insemination, the zygotes (with two pronuclei, both male and female) were cleaned and transferred to different treatments of KSOM culture medium, specific groups are as follows:

Control group: the basic development culture medium in vitro of mouse embryos was added to the special small dish for embryo culture, 2 droplets were made, 50 μL of culture medium per droplets, covered with paraffin oil, pre-balanced in an incubator with 37° C., 5% CO₂ saturated humidity for more than 6 hours, and the embryos were placed in well-balanced culture droplets, with 25˜35 pieces per droplets;

Treatment group 1: the development culture medium in vitro of mouse embryos containing 50 μmol/L of tributyrin was added to the special dish for embryo culture, 2 droplets were made, 50 μL of culture medium per droplets, covered with paraffin oil, pre-balanced in an incubator with 37° C., 5% CO₂ saturated humidity for more than 6 hours, and the embryos were placed in well-balanced culture droplets, with 25˜35 pieces per droplets;

Treatment group 2: the development culture medium in vitro of mouse embryos containing 100 μmol/L of tributyrin was added to the special dish for embryo culture, 2 droplets were made, 50 μL of culture medium per droplets, covered with paraffin oil, pre-balanced in an incubator with 37° C., 5% CO₂ saturated humidity for more than 6 hours, and the embryos were placed in well-balanced culture droplets, with 25˜35 pieces per droplets;

Treatment group 3: the development culture medium in vitro of mouse embryos containing 200 μmol/L of tributyrin was added to the special dish for embryo culture, 2 droplets were made, 50 μL of culture medium per droplets, covered with paraffin oil, pre-balanced in an incubator with 37° C., 5% CO₂ saturated humidity for more than 6 hours, and the embryos were placed in well-balanced culture droplets, with 25˜35 pieces per droplets;

The above different treatment groups were incubated in an incubator with 37° C., 5% CO₂ saturated humidity for 24 h, 48 h, 72 h and 96 h, corresponding to the 2-cell, 4-cell, morula and blastocyst stages of embryonic development, respectively, and the embryonic development was observed. The results are shown in Table 1 and FIG. 1 below.

TABLE 1
effect of tributyrin on development in vitro of mouse embryos

| Treatment | | | The rate of |
|---|---|---|---|
| group | The rate of 2-cell | The rate of 4-cell | blastocyst |
| 0 μmol/L | 96.13 ± 3.22% | 96.13 ± 3.09% | 69.53 ± 4.08%^{a} |
| 50 μmol/L | 96.58 ± 0.83% | 95.57 ± 0.57% | 78.37 ± 8.33%^{ab} |
| 100 μmol/L | 96.91 ± 0.76% | 96.57 ± 0.37% | 86.07 ± 16.53%^{b} |
| 200 μmol/L | 96.63 ± 0.5% | 96.46 ± 0.5% | 80.23 ± 10.70%^{ab} |

Note: different letters of the shoulder label of the same column of data represent significant differences in the same column (P<0.05), and no letters or the inclusion of the same letters indicate no significant differences (P>0.05).

As can be seen from the results in FIG. 1 and Table 1, the number of cells in the treatment group increased significantly compared to the control group (FIG. 1A), the rate of blastocyst was increased in the treatment group 1 compared with the control group, but the effect was not significant (78.37±8.33% vs 69.53±4.08%, P>0.05), treatment group 2 could significantly increase the rate of blastocyst compared with the control group (86.07±16.53% vs 69.53±4.08%, P<0.05), the rate of blastocyst was increased in the treatment group 3 compared with the control group, but the effect was not significant (80.23±10.70% vs 69.53±4.08%, P>0.05), the above results show that 50˜200 μmol/L of tributyrin can promote the development in vitro of mouse embryos and increase the rate of blastocyst; Furthermore, 100 μmol/L of tributyrin can significantly promote the development of in vitro mouse embryos and significantly increase the rate of blastocyst.
wherein, the data statistics and processing methods are as follows:

The rate of cleavage (%)=number of cleavage embryos/number of fertilized eggs with male and female pronuclei×100%

The rate of blastocyst (%)=number of blastocysts/number of cleavage embryos×100%

### Example 3: Effect of Tributyrin on Improve the Quality of Development In Vitro of Mouse Embryos

According to the experimental results in the above example 2, it can be seen that 100 μmol/L of tributyrin can significantly improve the rate of mouse blastocyst. Therefore, exemplically, experiments are carried out in the experimental group of this example using culture medium added 100 μmol/L of tributyrin, specifically, the experiments are divided into the following two groups:

Control group: the basic development culture medium in vitro of mouse embryos was added to the special small dish for embryo culture, 2 droplets were made, 50 μL of culture medium per droplets, covered with paraffin oil, pre-balanced in an incubator with 37° C., 5% CO₂ saturated humidity for more than 6 hours, and the embryos were placed in well-balanced culture droplets, with 25˜35 pieces per droplets;

Experimental group: the development culture medium in vitro of mouse embryos containing 100 μmol/L of tributyrin was added to the special dish for embryo culture, 2 droplets were made, 50 μL of culture medium per droplets, covered with paraffin oil, pre-balanced in an incubator with 37° C., 5% CO₂ saturated humidity for more than 6 hours, and the embryos were placed in well-balanced culture droplets, with 25˜35 pieces per droplets.

### (1) Detection of Reactive Oxygen Species (ROS) in Mouse Embryos

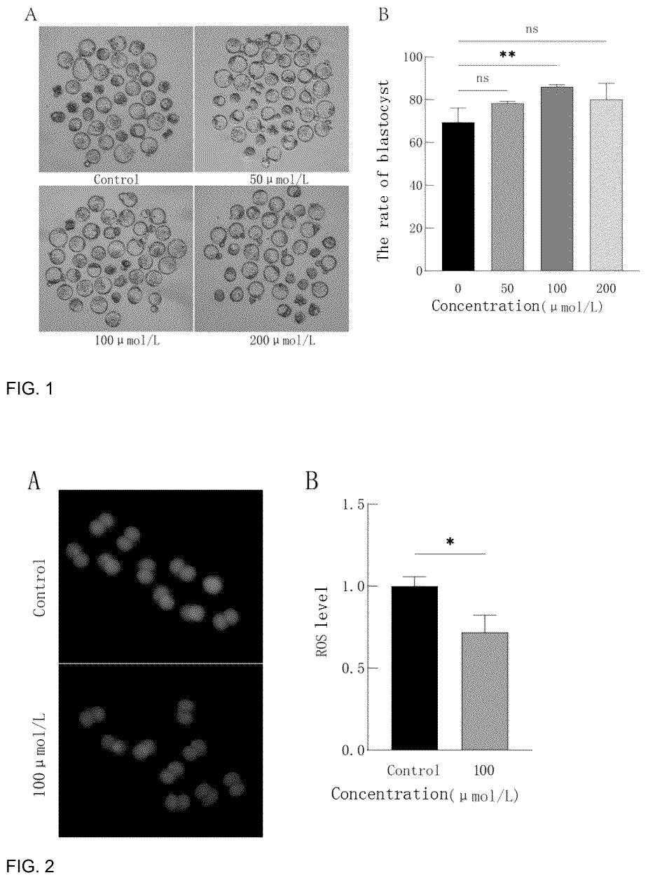

Referring to the steps of development culture in vitro of example 2, development culture in vitro was carried out on the above two treatment groups; according to the instructions of the reactive oxygen species detection kit (Biyuntian, S0033S), DCFH-DA was diluted to a working solution concentration of 5 UM with KSOM culture medium. After the embryos of the control group and the experimental group at the 2-cell stage were collected, washed twice in the pre-warmed culture medium, and then transferred to the microdroplets containing the DCFH-DA working solution, incubated in the dark at 37° C., 5% CO₂ incubator for 30 min, after washing with clean KSOM culture medium for 2˜3 times, it was placed under an inverted fluorescence microscope for observation and photography. Image J software was used to analyze the average fluorescence intensity of ROS in embryos. The results are shown in FIG. 2.

As can be seen from FIG. 2, the average fluorescence intensity of ROS in mouse embryos decreased after treatment with 100 μmol/L of tributyrin, and the results showed that 100 μmol/L of tributyrin significantly reduced the ROS level in

### (2) Detection of Mitochondrial Membrane Potential

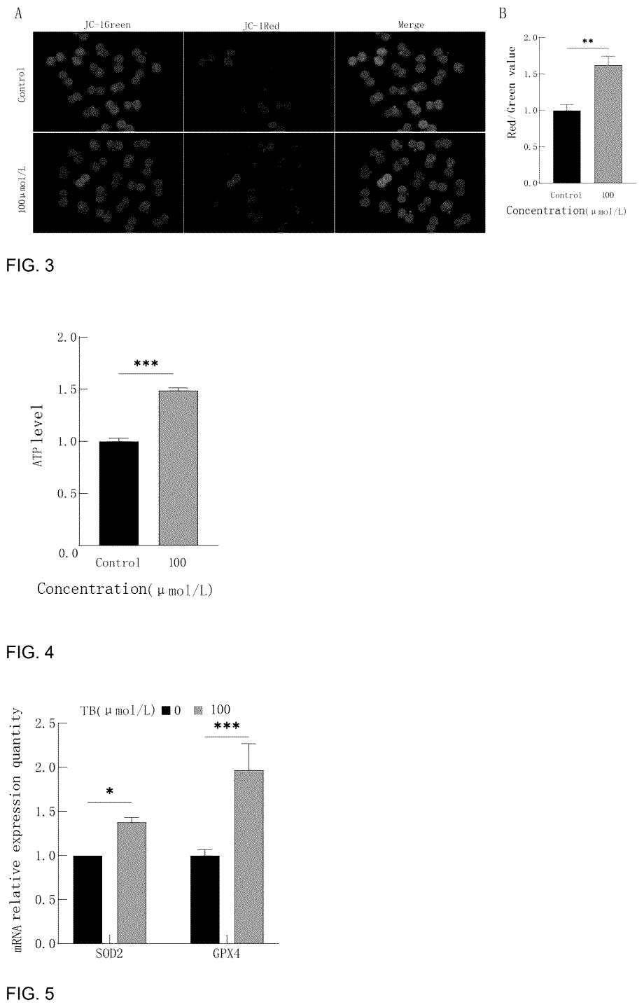

Referring to the steps of development culture in vitro of example 2, development culture in vitro was carried out on the above two treatment groups; according to the instructions of the mitochondrial membrane potential J C-1 detection kit (Biyuntian, C2006), the embryos of the control group and the experimental group at the 2-cell stage were transferred into 1×J C-1 staining working solution, incubated in the dark at 37° C., 5% CO₂ incubator for 30 min, then wash the embryos with 1×J C-1 staining buffer, it was placed under an inverted fluorescence microscope for observation and photography. Image J software was used to analyze the average fluorescence intensity of J C-1 red and J C-1 green in embryos. The results are shown in FIG. 3.

As can be seen from FIG. 3, the relative fluorescence intensity of J C-1 red/green in mouse embryos was enhanced after treatment with 100 μmol/L of tributyrin, and the results showed that 100 μmol/L of tributyrin significantly improved the mitochondrial membrane potential level.

### (3) Detection of ATP Level in Mouse Embryos

Referring to the steps of development culture in vitro of example 2, development culture in vitro was carried out on the above two treatment groups; according to the instructions of the ATP detection kit (Biyuntian, S0027), the embryos of the control group and the experimental group at the 2-cell stage were tested for ATP. Add 16 μL of ATP detection lysate to 200 μL centrifuge tube and transfer into 40 pieces 2-cell stage embryos. Take the centrifuge tube with tweezers, place it in liquid nitrogen for freezing, then place it in room temperature H₂O for thawing after the liquid at the bottom of the centrifuge tube solidifies, and so on twice. Perform rapid high-speed vortexing, centrifuge after the vortex to get the liquid off the bottom of the tube without bubbles, and place the sample on ice after centrifugation. 64 μL of ATP working solution (ATP detection reagent diluent: ATP detection reagent=4:1) was added to the tube, vortex and centrifuged to the bottom of the tube, quickly transferred to a black 96-well plate with a multifunctional microplate reader for detection. The results are shown in FIG. 4.

As can be seen from FIG. 4, the ATP level in mouse embryos was significantly increased after treatment with 100 μmol/L of tributyrin, and the results showed that 100 μmol/L of tributyrin significantly increased the ATP content in embryos.

### (4) Detection of Antioxidant Gene Expression in Mouse Embryos

Referring to the steps of development culture in vitro of example 2, development culture in vitro was carried out on the above two treatment groups; embryos from the control group and the experimental group at the 2-cell stage were obtained, the high-purity RNA was extracted according to the instructions of the RNAprep Pure Total RNA Extraction Kit for Micro Samples (Tiangen, DP420). Reverse transcription was performed with the HiScript II Q RT SuperMix for Qrcr Reverse Transcription Kit (Novozan, R223) at 50° C., 15 min, 85° C., 5 s. PCR amplification was performed using 1.5 μL of cDNA, 10 μL of 2×ChamQ Universal SYBR Qpcr Master Mix (Novozam, Q711), 0.5 μL of forward and reverse primers, and 7.5 μL of H₂O. Reaction conditions: 95° C., (pre-denaturation) 3 min; 95° C. (denaturation) for 10 s; 55° C. (annealed) for 30 s; 40 cycles (PCR amplification); the fluorescent signal was collected at 55° C. extension, the RPLPO gene was used as an internal control, and the expression was calculated using the 2^{-ΔΔct} formula, wherein, the primer sequences of each gene are shown in Table 2 below:

TABLE 2
primer sequence list

| | | | Accession |
|---|---|---|---|
| Gene | Primer sequences (5′→3′) | Length | number |
| RPLP0 | F: AGGCCCTGCACTCTCGCTTTC(SEQ ID NO: 1) | 189 | NM_001206359.1 |
| | R: GGGTACCCGATCTGCAGACACAC(SEQ ID NO: 2) | | |
| SOD2 | F: CAACCAGTTTGGACATCAGGAAAA(SEQ ID NO: 3) | 132 | NM_001190422.1 |
| | R: GCATTTGCGCCATTCACCTC(SEQ ID NO: 4) | | |
| GPX4 | F: ATGCCCGATATGCTGAGTGT(SEQ ID NO: 5) | 235 | NM_001037741.4 |
| | R: GCTAGAGATAGCACGGCAGG(SEQ ID NO: 6) | | |

The results are shown in FIG. 5.

As can be seen from FIG. 5, the expression of antioxidant genes (SOD2, GPX4) in mouse embryos was significantly increased after treatment with 100 μmol/L of tributyrin, and the results showed that 100 μmol/L of tributyrin significantly promoted the expression of antioxidant genes in embryos.

### (5) Detection of DNA Methylation Level

Referring to the steps of development culture in vitro of example 2, development culture in vitro was carried out on the above two treatment groups; embryos from the control group and the experimental group at the 2-cell stage were obtained, in order to detect the DNA methylation level in mouse embryos, the cells were first fixed with paraformaldehyde, TritonX-100 enhanced the permeability of the cell membrane to facilitate the binding of antibody antigens. After acidification and base neutralization, the non-specific protein is blocked with 2% BSA, followed by incubation with the corresponding primary antibody and fluorescently labeled secondary antibody. The specific methods and steps are as follows:

\textcircled{1} Mouse embryos fixation and permeabilization: mouse embryos were placed in a washing solution (PBS containing 0.1% Tween-20 and 0.01% Triton X-100) for rapid washing for 1 time, then piped into the fixed permeabilization solution (PBS containing 4% paraformaldehyde and 0.5% Triton) and treated at room temperature for 50 min. After the solidification is completed, the embryos are transferred to the washing solution and washed 3 times, 2 min each time.

\textcircled{2} Acidification: 4 N hydrochloric acid acidification treatment for 15 min.

\textcircled{3} Neutralization: pH 8.0, 100 mM Tris-HCL neutralization for 20 min.

\textcircled{4} Blocking: transfer the cleaned embryos to the blocking solution (cleaning solution containing 2% BSA, use immediately after preparation) and blocking at room temperature for 1 h.

\textcircled{5} Primary antibody incubation: after the blocking is completed, the embryos are directly transferred to the 5 hmC primary antibody (Active Motif, 91309, 1:200) diluted in the blocking solution, and incubated at 4° C. overnight. The next day, wash 3 times with cleaning solution, 8 min each time.

\textcircled{6} Secondary antibody incubation: after the cleaning is completed, the embryos are transferred the secondary antibody diluted in the blocking solution, incubate at 37° C. wet box in the dark for 1 h. After the incubation is completed, wash 3 times with cleaning solution, 8 min each time.

\textcircled{7} DNA staining: place the embryos in DAPI and incubated at room temperature in the dark for 5 min. After the incubation is completed, wash 3 times with the cleaning solution, 3 min each time.

\textcircled{8} Cover-slip: selecting a square area the size of a coverslip on a clean glass slide, add a small amount of support agent dropwise around it, then drop about 5 μL of quenching cover-slip solution in the center, quickly transfer the cleaned embryos to make it evenly dispersed in the liquid, quickly cover the coverslip, and gently press the coverslip under the stereoscope until the embryo becomes 1.2 times larger.

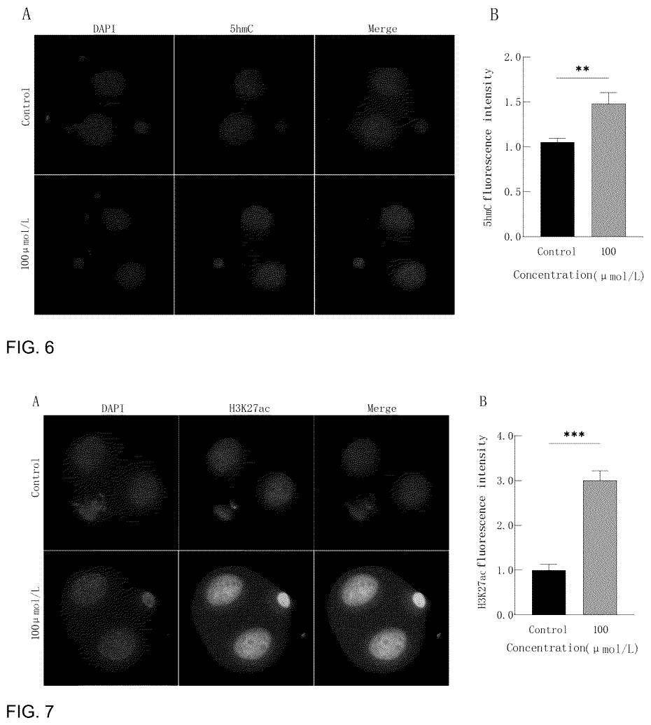

The results are shown in FIG. 6.

As can be seen from FIG. 6, the 5 hmC level in mouse embryos was significantly increased after treatment with 100 μmol/L of tributyrin, and the results showed that 100 μmol/L of tributyrin increased the 5 hmC level in mouse embryos.

### (6) Detection of Histone Acetylation Level

Referring to the steps of development culture in vitro of example 2, development culture in vitro was carried out on the above two treatment groups; embryos from the control group and the experimental group at the 2-cell stage were obtained, the histone H3K27ac level was detected by immunofluorescence. The specific methods and steps are as follows:

\textcircled{1} Mouse embryos fixation and permeabilization: mouse embryos were placed in a washing solution (PBS containing 0.1% Tween-20 and 0.01% Triton X-100) for rapid washing for 1 time, then piped into the fixed permeabilization solution (PBS containing 4% paraformaldehyde and 0.5% Triton) and treated at room temperature for 50 min. After the solidification is completed, the embryos are transferred to the washing solution and washed 3 times, 2 min each time.

\textcircled{2} Blocking: transfer the cleaned embryos to the blocking solution (cleaning solution containing 2% BSA, use immediately after preparation) and blocking at room temperature for 1 h.

\textcircled{3} Primary antibody incubation: after the blocking is completed, the embryos are directly transferred to the H3K27ac primary antibody (Cell Signaling Technology, A7253, 1:200) diluted in the blocking solution, and incubated at 4° C. overnight. The next day, wash 3 times with cleaning solution, 8 min each time.

\textcircled{4} Secondary antibody incubation: after the cleaning is completed, the embryos are transferred the secondary antibody diluted in the blocking solution, incubate at 37° C. wet box in the dark for 1 h. After the incubation is completed, wash 3 times with cleaning solution, 8 min each time.

\textcircled{5} DNA staining: place the embryos in DAPI and incubated at room temperature in the dark for 5 min. After the incubation is completed, wash 3 times with the cleaning solution, 3 min each time.

\textcircled{6} Cover-slip: selecting a square area the size of a coverslip on a clean glass slide, add a small amount of support agent dropwise around it, then drop about 5 μL of quenching cover-slip solution in the center, quickly transfer the cleaned embryos to make it evenly dispersed in the liquid, quickly cover the coverslip, and gently press the coverslip under the stereoscope until the embryo becomes 1.2 times larger.

The results are shown in FIG. 7.

As can be seen from FIG. 7, the histone H3K27ac level in mouse embryos was significantly increased after treatment with 100 μmol/L of tributyrin, and the results showed that 100 μmol/L of tributyrin increased the histone H3K27ac level in

To sum up, in the present disclosure, the tributyrin is applied as an additive to the development culture medium of mouse embryos in vitro for the first time, the tributyrin can significantly increase the rate of blastocyst, and reduce the ROS content in the embryos, improve the mitochondrial membrane potential in the embryos, increase the ATP level and the expression of antioxidant genes in the embryos, improve the DNA methylation and histone modification level in the embryos, promote the embryos development in vitro.

It should be noted that the above examples all belong to the same invention concept, and the descriptions of each examples have their own emphasis, and where the descriptions are not exhaustive in the individual examples, reference can be made to the descriptions in other examples.

The above examples only express the examples of the present disclosure, and its description is more specific and detailed, but it cannot be understood as a restriction on the scope of the disclosure. It should be noted that for a person skilled in the art, a number of deformations and improvements can be made without departing from the conception of the present disclosure, which are within the scope of protection of the present disclosure. Therefore, the scope of protection of the disclosure shall be subject to the attached claims.

1-3. (canceled)

4. An in vitro embryonic development culture medium for promoting embryo development, wherein the culture medium comprises tributyrin as an additive.

5. The in vitro embryonic development culture medium for promoting embryo development according to claim 4, wherein the concentration of the additive is 50 to 200 μmol/L.

6. The in vitro embryonic development culture medium for promoting embryo development according to claim 5, wherein the concentration of the additive is 100 μmol/L.

7. The in vitro embryonic development culture medium for promoting embryo development according to claim 4, wherein the culture medium further comprises potassium simplex optimized medium (KSOM) basal culture medium.
